SEQUENCE LISTING (5 sequences)

SEQ ID NO: 1 - DNA, 22 bp, Artificial Sequence
  Primer
ccgcggaaagcttagacccaag

SEQ ID NO: 2 - DNA, 22 bp, Artificial Sequence
  Primer
gctagcttcctgccctgcctct

SEQ ID NO: 3 - DNA, 21 bp, Artificial Sequence
  Primer
acctgaaagcgaaagggaaac

SEQ ID NO: 4 - DNA, 23 bp, Artificial Sequence
  Primer
cacccatctctctccttctagcc

SEQ ID NO: 5 - DNA, 24 bp, Artificial Sequence
  Primer
agctctctcgacgcaggactcggc

(19) United States
(12) Patent Application Publication
(10) Pub. No.: US 2010/0172871 A1
(43) Pub. Date: Jul. 8, 2010

(54) Muller Cell Specific Gene Therapy

(76) Inventors: John G. Flannery, Berkeley, CA (US); Kenneth P. Greenberg, Oakland, CA (US)

Correspondence Address: BOZICEVIC, FIELD & FRANCIS LLP, 1900 UNIVERSITY AVENUE, SUITE 200, EAST PALO ALTO, CA, 94303, US

(21) Appl. No.: 11/884,418

(22) PCT Filed: Feb. 16, 2006
(86) PCT No.: PCT/US06/05801

§ 371 Date: Mar. 10, 2009

Related U.S. Application Data
(60) Provisional application No. 60/654,640, filed on Feb. 17, 2005.

(51) Int. Cl. A61K 48/00; A61P 27/02

(52) U.S. Cl. 424/93.2

(57) ABSTRACT

The present invention provides methods and compositions for the treatment of disease of the eye, such as retinitis pigmentosa (RP) and glaucoma, by delivery of a transgene encoding a therapeutic polypeptide, such as glial cell-derived neurotrophic factor (GDNF), specifically to Müller glial cells using a gene delivery vector. In one embodiment, the gene delivery vector is a pseudotyped retroviral vector, particularly a lentiviral vector.

### CROSS-REFERENCE

This application claims the benefit of U.S. Provisional Patent Application No. 60/654,640, filed Feb. 17, 2005, which application is incorporated herein by reference in its entirety.

### GOVERNMENT RIGHTS

This invention was made with government support under federal grant nos. EY13533 awarded by the National Institutes of Health. The United States Government may have certain rights in this invention.

### BACKGROUND OF THE INVENTION

Eye diseases represent a significant health problem in the U.S. and around the world. A wide variety of eye diseases can cause visual impairment, including for example, macular degeneration, diabetic retinopathies, inherited retinal degeneration such as retinitis pigmentosa, glaucoma, retinal detachment or injury and retinopathies (whether inherited, induced by surgery, trauma, a toxic compound or agent, or, photically).

The retina can be particularly affected by in eye disease. The retina, a structure located at the back of the eye, is a specialized light-sensitive tissue that contains photoreceptor cells (rods and cones) and neurons connected to a neural network for the processing of visual information. This information is sent to the brain for decoding into a visual image.

The retina depends on cells of the adjacent retinal pigment epithelium (RPE) for support of its metabolic functions. Photoreceptors in the retina, perhaps because of their huge energy requirements and highly differentiated state, are sensitive to a variety of genetic and environmental insults. The retina is thus susceptible to a variety of diseases that result in visual loss or complete blindness.

An example of such a disease is the blinding disease Retinitis Pigmentosa (RP), which is a candidate for a neuroprotective treatment strategy with techniques of gene therapy. RP is a heterogeneous group of inherited disorders, each characterized by the degeneration of rods, cones, and the RPE in the human retina. The degenerative process and photoreceptor neuronal cell death generally takes place over the course of many years. Mutations which cause RP have been identified in many of the rod and cone photoreceptor genes involved in the phototransduction cascade, including those for rhodopsin, alpha- and beta-subunits of rod cGMP-phosphodiesterase, alpha-subunit of the rod cGMP-gated channel, arrestin, and RP GTPase regulator (Phelan, et al. (2000) Mol. Vis. 6: (2), 116-124). Other RP causing mutations have been detected in genes that code for proteins involved in photoreceptor and RPE structure and metabolism, including RDS, ROM1, cellular retinaldehyde binding protein, RPE65, myosin VIIA, and ABCA4 (Phelan, et al. (2000) Mol. Vis. 6: (2), 116-124). Rhodopsin mutations are most prevalent and account for approximately 10 percent of all cases. Many diseases are monogenic, generated by one mutation in one gene, but this heterogeneous group of diseases which are collectively called RP is unusual in that so many different mutations produce a similar disease phenotype. For RP therefore, it may be important to assess the utility of non-gene specific forms of therapy that could be employed against a variety of RP disease types.

Other diseases of the eye, such as glaucoma, are also major public health problems in the United States. Glaucoma is not a uniform disease but rather a heterogeneous group of disorders that share a distinct type of optic nerve damage that leads to loss of visual function. The disease is manifest as a progressive optic neuropathy that, if left untreated, leads to blindness. Glaucoma can involve several tissues in the front and back of the eye. Commonly, but not always, glaucoma begins with a defect in the front of the eye. Fluid in the anterior portion of the eye, the aqueous humor, forms a circulatory system that brings nutrients and supplies to various tissues. Aqueous humor enters the anterior chamber via the ciliary body epithelium (inflow), flows through the anterior segment bathing the lens, iris, and cornea, and then leaves the eye via specialized tissues known as the trabecular meshwork and Schlemm's canal to flow into the venous system. Intraocular pressure is maintained vis-a-vis a balance between fluid secretion and fluid outflow. Almost all glaucomas are associated with defects that interfere with aqueous humor outflow and, hence, lead to a rise in intraocular pressure. The consequence of this impairment in outflow and elevation in intraocular pressure is that optic nerve function is compromised. The result is a distinctive optic nerve atrophy, which clinically is characterized by excavation and cupping of the optic nerve, indicative of loss of optic nerve axons.

Primary open-angle glaucoma, the most prevalent form of glaucoma, is, by convention, characterized by relatively high intraocular pressures believed to arise from a blockage of the outflow drainage channel or trabecular meshwork in the front of the eye. However, another form of primary open-angle glaucoma, normal-tension glaucoma, is characterized by a severe optic neuropathy in the absence of abnormally high intraocular pressure. Patients with normal-tension glaucoma have pressures within the normal range, albeit often in the high normal range. Both these forms of primary open-angle glaucoma are considered to be late-onset diseases in that, clinically, the disease first presents itself around midlife or later. However, among African-Americans, the disease may begin earlier than middle age. In contrast, juvenile open-angle glaucoma is a primary glaucoma that affects children and young adults. Clinically, this rare form of glaucoma is distinguished from primary open-angle glaucoma not only by its earlier onset but also by the very high intraocular pressure associated with this disease.

Primary open-angle glaucoma can be insidious. It usually begins in midlife and progresses slowly but relentlessly. If detected, disease progression can frequently be arrested or slowed with medical and surgical treatment. However, without treatment, the disease can result in absolute irreversible blindness. In many cases, even when patients have received adequate treatment (e.g., drugs to lower intraocular pressure), optic nerve degeneration and loss of vision continues relentlessly.

Angle-closure glaucoma is a mechanical form of the disease caused by contact of the iris with the trabecular meshwork, resulting in blockage of the drainage channels that allow fluid to escape from the eye. This form of glaucoma can be treated effectively in the very early stages with laser surgery. Congenital and other developmental glaucomas in children tend to be severe and can be very challenging to treat successfully. Secondary glaucomas result from other ocular diseases that impair the outflow of aqueous humor from the eye and include pigmentary glaucoma, pseudoexfoliative glaucoma, and glaucomas resulting from trauma and inflammatory diseases. Blockage of the outflow channels by new blood vessels (neovascular glaucoma) can occur in people with retinal vascular disease, particularly diabetic retinopathy.

Neurotrophic factors are known to modulate neuronal growth during development to maintain existing cells and to allow recovery of injured neuronal populations. Observations of retinal neurons during development (Crespo et al., (1985) Brain Research 351: (1), 129-134) suggest that correct synaptic connections are reinforced by trophic factors, while cells that make inappropriate connections and do not receive trophic support undergo apoptosis. Hence, it has long been hypothesized that if the removal of neurotrophic factors from the cellular environment can stimulate cell death then adding exogenous trophic factors may have neuroprotective effects in the retina (Faktorovich, et al. (1990) Nature 347: (6288), 83-86).

GDNF was first described as a stimulant of survival of dopaminergic neurons in-vitro (Lin, et al. (1993) Science 260: (5111), 1130-1132) and was found to belong to the transforming growth factor-beta superfamily. Shortly after its discovery, it was demonstrated to have protective effects in in-vivo models of Parkinson's Disease (Kaddis, et al. (1996) Cell Tissue Res. 286: (2), 241-247; Gash, et al. (1996) Nature 380: (6571), 252-255; Choi-Lundberg, et al. (1997) Science 275: (5301), 838-841), on dorsal root ganglion neurons (Matheson, et al. (1997) J. Neurobiol. 32: (1), 22-32), and on motor neurons during development (Oppenheim, et al. (1995) Nature 373: (6512), 344-346). GDNF interacts with a specific cell-surface receptor, GFRA1 (Jing, et al. (1996) Cell 85:(7), 1113-1124; Treanor, et al. (1996) Nature 382: (6586), 80-83), and its biological effects are mediated through the interaction of GDNF, GFRA1, and a tyrosine kinase receptor, RET (Takahashi, et al. (1987) Mol Cell Biol 7: (4), 1378-1385). Both GDNF and its receptors are synthesized in the retina (Jing, et al. (1996) Cell 85: (7), 1113-1124; Nosrat, et al. (1996) Cell Tissue Res. 286: (2), 191-207; Pachnis, et al. (1993) Development 119: (4), 1005-1017). GDNF protein have been examined in photoreceptors in the Pde6b^{-/-} (rd) mouse (Frasson, et al. (1999) Invest. Opthalmol. Vis. Sci. 40: (11), 2724-2734), in photoreceptor outer segment collapse in-vitro (Carwile, et al. (1998) Exp. Eye Res. 66: (6), 791-805), and in mouse photoreceptors in-vitro (Jing, et al. (1996) Cell 85: (7), 1113-1124).

A great deal of the progress made in addressing the important clinical problems of conditions such as RP and glaucoma has depended on advances in research on photoreceptor cell biology, molecular biology, molecular genetics, and biochemistry over the past two decades. Animal models of hereditary retinal disease have been vital in helping unravel the specific genetic and biochemical defects that underlie abnormalities in human retinal diseases. It now seems clear that both genetic and clinical heterogeneity underlie many hereditary retinal diseases.

A number of neurotrophins have been tested for their ability to support photoreceptor survival in various models of retinal degeneration (Frasson, et al. (1999) Invest. Opthalmol. Vis. Sci. 40: (11), 2724-2734; Cayouette, et al. (1997) Hum. Gene. Ther. 8: (4), 423-430; LaVail, et al. (1998) Invest. Opthalmol. Vis. Sci. 39: (3), 592-602; Lau, et al. (2000) Invest. Opthalmol. Vis. Sci. 41: (11), 3622-3633; Jablonski, et al. (2000) J. Neuroscience 20: (19), 7149-7157). Photoreceptors have high oxygen and nutrient demands and must maintain a complex equilibrium of extracellular and intracellular ions for phototransduction. This makes rods and cones particularly susceptible to genetic, structural, and biochemical insults (Travis (1998) Am. J. Hum. Genet. 62: (3), 503-508; Stone, et al. (1999) Prog. Retin. Eye. Res. 18: (6), 689-735). Disturbances in the visual cycle appear to trigger apoptotic cell death in photoreceptors.

Substantial effort in retinal degeneration research has focused on the therapeutic effect of neurotrophins as a general protective strategy to slow the progression of degeneration. Specific gene therapies, such as antisense or ribozymes (Lewin, et al. (1998) Nat. Med. 4: (8), 967-971), which work to eliminate mutant mRNA of the affected gene, have promise for treating dominant forms of RP. Unfortunately, different ribozyme or antisense therapies must be designed for each specific mutation. Gene replacement may be used as a therapy for recessive forms of RP (Lem, et al. (1992) Proc Natl Acad Sci USA 89: (10), 4422-4426; Travis, et al. (1992) Neuron 9: (1), 113-119; Bennett, et al. (1996) Nat. Med. 2: (6), 649-654), but it cannot readily treat the majority of RP patients. An alternative to these gene-specific therapies is generalized survival factor therapy that does not target the mutant gene product, but alters the photoreceptor environment in a manner promoting cell survival. The aim is to slow the rate of cell death therefore prolonging the period of useful vision for patients.

LaVail, Steinberg, and colleagues pioneered this field by testing many different survival factors in rat models of photoreceptor degeneration (Faktorovich, et al. (1990) Nature 347: (6288), 83-86; Faktorovich, et al. (1992) J. Neurosci. 12: (9), 3554-3567; LaVail, et al. (1992) Proc Natl. Acad. Sci. USA 89: (23), 11249-11253; see also U.S. Pat. No. 5,667,968). They noted a slowing of photoreceptor cell death with direct protein injections of different growth factors or neurotrophic agents, including basic fibroblast growth factor (FGF2), CNTF, and BDNF. However, prolonged rescue of photoreceptor degeneration by intraocular injection of protein has been difficult to achieve because therapeutic proteins are continuously degraded in the body and lose biological activity over a short period of time. Theoretically, the rescue seen with protein injections could be sustained with repetitive delivery; however, repetitive injection of survival factors into the subretinal space is not a practical regimen for RP patients.

Gene delivery methods hold promise because photoreceptor cells, if properly transduced, can continually produce their own neurotrophic factor. One vector of interest for retinal gene therapy in humans is recombinant adeno-associated virus (rAAV) (Hauswirth, et al. (2000) Invest Opthalmol Visual Sci 41: (10), 2821-2826; see also WO 00/54813). When injected subretinally, rAAV delivers the gene of interest to photoreceptors and to the RPE (Acland, et al. (2001) Nature Genetics 28: (1), 92-95). Additionally, recombinant AAV vectors are not associated with any known human disease. Moreover, recent improvements in rAAV production have made manufacturing of high titer gene transfer vector easily attainable. In a previous study using AAV to transduce the retina, the expression levels increased progressively after 1 week post-injection and plateau at approximately 5 weeks post-injection (McGee Sanftner, et al. (2001) Mol. Ther. 3: (5 Pt 1), 688-696).

Despite advances in the field, the optimal neurotrophic factor for delivery to the retina and treatment eye diseases has not yet been identified in the art. For example, while the neurotrophic growth factors (e.g., fibroblast growth factors), appear promising (see, e.g., WO 00/54813), there are concerns that such factors may also promote new blood vessel formation, placing a patient at risk of, for example, a macular degenerative-type disorder, particularly in individuals who are susceptible macular degeneration. Furthermore, while some therapies rescue the cells from cell death, preserving the physiology of the cell, little success has been reported to date in the protection of cells in a manner that preserves the electrophysiologic response of the retina to light. The present invention solves these problems.

### SUMMARY OF THE INVENTION

The present invention provides methods and compositions for the treatment of disease of the eye, such as retinitis pigmentosa (RP) and glaucoma, by delivery of a transgene encoding a therapeutic polypeptide, such as glial cell-derived neurotrophic factor (GDNF), specifically to Müller glial cells using a gene delivery vector. In one embodiment, the gene delivery vector is a pseudotyped retroviral vector, particularly lentiviral vector.

In one aspect, the invention features a method for treating or preventing diseases of the eye, comprising, administering to a subject a Müller cell specific retroviral gene delivery vector which directs the expression of a therapeutic polypeptide in the Müller cell, such that said disease of the eye is treated or prevented. In some embodiments, the disease of the eye is macular degeneration, diabetic retinopathy, retinitis pigmentosa, glaucoma, a surgery-induced retinopathy, retinal detachment, a photic retinopathy, a toxic retinopathy, or a trauma-induced retinopathy. In such embodiments, the vector may be administered to the eye of the subject, such as by intraocular administration, or by subretinal administration. In some embodiments, the retroviral gene delivery vector is a lentiviral vector. In further embodiments, the lentiviral vector is pseudotyped with a Ross River Virus glycoprotein.

In some embodiments, the therapeutic polypeptide is a neurotrophic factor. In further embodiments, the neurotrophic factor is FGF, NGF, BDNF, CNTF, NT-3, or, NT-4. In other embodiments, the therapeutic polypeptide is an anti-angiogenic factor. In further embodiments, the anti-angiogenic factor is soluble Flt-1, PEDF, soluble Tie-2 receptor, or, a single chain anti-VEGF antibody. In yet other embodiments, the therapeutic polypeptide is a neurotrophic factor. In further embodiments, the neurotrophic factor is GDNF, FGF, NGF, BDNF, CNTF, NT-3, or, NT-4.

In another aspect, the present invention provides a kit adapted for use in the subject methods, the kit comprising a sterile container containing a Müller cell specific retroviral gene delivery vector adapted for expression of a therapeutic polypeptide in an eye of the subject. In some embodiments, the kit comprises a sterile needle adapted for injection of the recombinant gene delivery vector into an eye of the subject.

These and other objects, advantages, and features of the invention will become apparent to those persons skilled in the art upon reading the details of the invention as more fully described below.

### BRIEF DESCRIPTION OF THE DRAWINGS

The invention is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity. Included in the drawings are the following figures:

FIG. 1 is 3-D schematic drawing of the close anatomical relationship between a Müller cell and all classes of retinal neurons.

FIG. 2 is schematic of additional HIV-1 based vectors containing the HIV-1 central polypurine tract (CPPT), promoter, enhanced green fluorescent protein (eGFP) cDNA, and woodchuck hepatitis virus posttranscriptional regulatory element (WPRE). Promoters include; human cytomegalovirus (CMV), human ubiquitin-C, hybrid CMV/chicken beta-actin (CAG), mouse CD44, mouse glial fibrillary acidic protein (GFAP), and mouse vimentin (VIM). Abbreviations: long terminal repeat (LTR), splice donor (SD), packaging signal (ψ), self-inactivating long terminal repeat (SIN LTR).

FIG. 3 is a schematic of an in vivo electroporation injection and current application protocol.

FIG. 4 is a series of schematic maps of pFmGFAP(FL)GW and pFUGW transfer vector plasmids (panel A) and pFhGFAPGW and pGfa2-cLac human GFAP promoted plasmids (Panel B).

FIG. 5 is a Q-PCR Amplification plot of pCS-CG plasmid (panel A), and standard curve (panel B) generated by plotting threshold cycle (Ct) against number of vector DNA molecules.

FIG. 6 is a series of images showing GFP expression at injection site (GFP on left, bright field on right) in flatmount retina injected intravitreally with VSV-mGFAP(FL)-GFP LV vector.

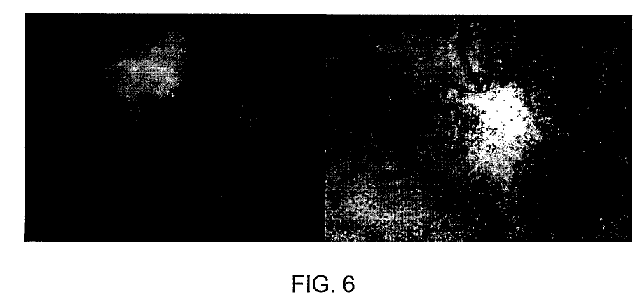

FIG. 7 is a series of photographs showing Müller cells expressing GFP Following intravitreal injection of LV-mGFAP-GFP (panel A), deconvolution image slice showing individual Müller cells expressing LV delivered GFP spanning entire thickness of retina (panel B), high magnification confocal image of GFP positive Müller cell nuclei and apical processes in vivo (panel C), and low magnification confocal image of GFP positive Müller cell nuclei and apical processes in vivo (panel D).

FIG. 8 is a section from retina in FIG. 8, panel D, showing Müller cells stained with α-vimentin antibody.

FIG. 9 is an image of Widefield Retcam II fundus images showing extent of GFP expression (top) and brightfield image (bottom) 10 days following subretinal injection of Müller specific LV vector.

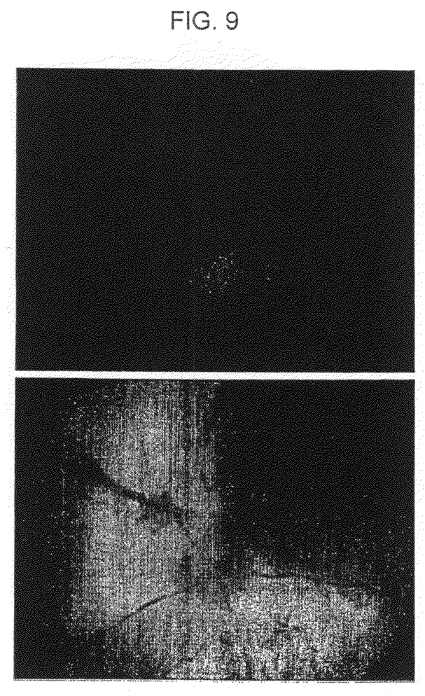

FIG. 10 is an image of cultured Müller cells stained positive for GFAP (left panel) and Vimentin (right panel).

FIG. 11 is a series of graphs showing a comparison of Müller cell transduction by RRV and VSV pseudotyped LV vectors (panel A) and relative transduction efficiency of RRV-LV vector in three cell lines (panel B).

FIG. 12 is a series of images showing that a combination of RRV pseudotyping and transcriptional targeting (CBA, mVIM, mGFAP) permits LV-GFP expression in cultured Müller cells.

FIG. 13 is an image of a GFP positive RPE layer after subretinal injection of RRV-CMV-GFP LV (4×10⁶ TU). High magnification inset shows individual GFP positive RPE cells.

FIG. 14 is a series of photographs of fluorescent fundus image showing widespread GFP expression in rat retina 1 week after subretinal injection of 3 μL VSV-CPA-GFP lentiviral vector (panel A), and fundus image of same rat under white light illumination (panel B). Arrows indicate small hemorrhage resulting from subretinal injection. Both images acquired with a Retcam II imaging system (Massie Research, Pleasanton, Calif.).

FIG. 15 is a series of images showing high magnification view of GFP positive photoreceptors of mouse retina injected subretinally with VSV-CMV-GFP LV vector at age P7 (panel A), lower magnification view (panel B) of the same retina shown in (panel A) where RPE and photoreceptors are seen expressing GFP. Expression of GFP restricted to the RPE layer in P14 mouse retina injected with VSV-CMV-GFP LV vector (panel C). Injection track mark shown (arrows) and evidence of immune response from autoflourescent macrophages bordering track mark (panel D). Low magnification view showing extent of GFP expression along entire length of the RPE (panel E).

FIG. 16 shows in vivo fluorescent fundus images of rat retinas injected subretinally with VSV.CD44.GFP (panel A) and VSV.CMV.GFP (panel B) LV vectors and intravitreal injection (panel C).

FIG. 17 shows high Müller cell transduction efficiency and detailed anatomy observed following LV vector mediated GFP delivery. The confocal image shows a SD rat retina (100 μm thick agarose section) 10 days after subretinal injection of VSV.CD44.GFP LV vector. ILM and branched fiber basket matrix of GFP positive Müller cells are seen at top of image.

FIG. 18 is a series of images showing LV vector delivered GFP expression in healthy and diseased retinas. Following VSV.CD44.GFP vector injection (panel A) GFP positive Müller cells are observed spanning the entire thickness of SD rat retina far from the injection site (scale bar represents 50 μm). Panel B shows Müller cell processes are surrounding DAPI-stained photoreceptor nuclei shown in blue. Panel C shows high magnification en face view of Müller cell fiber basket matrix at the OLM. Panel D shows GFP positive, panel E shows glutamine synthetase stained, and panel F shows merged Müller cells are disorganized likely as a result of subretinal injection procedure. Following VSV.GFAP.GFP vector injection, GFP positive (panel G) Müller cells are observed in the diseased S334Ter+/−retina (panel H) stained with a rhodopsin antibody, a Müller cell apical process (panel I, arrow head) is observed penetrating though the OLM into the subretinal space in the merged view.

FIG. 19 is a series of images showing Müller cells in the diseased retina. Reactive gliosis caused by subretinal injection procedure resulting in a large glial scar formation seen in cross section of S334Ter+/−rat retina injected with VSV.GFAP.GFP vector (panel A is GFP, panel B is GS, and paned C is merged).

FIG. 20 shows scotopic ERG recordings following LV vector injection. Example of dark adapted ERG traces from VSV.CD44.GFP vector and PBS injected eyes recorded 1 month post injection. No significant difference in b-wave amplitude is observed between vector injected (left panel) and PBS controls (right panel).

FIG. 21 is a schematic showing glial-neuronal interaction in the light-degenerated retina.

FIG. 22 is a series of schematics of a pTR-UPwGDNF map containing human GDNF cDNA (panel A) and a pFmGFAP(FL)GDNFW LV transfer vector (panel B).

FIG. 23 is map showing RRV envelope glycoprotein subunits.

FIG. 24 is pRRV-E2E1A(N218R) glycoprotein map.

### DETAILED DESCRIPTION OF THE INVENTION

The present invention provides methods and compositions for the treatment of disease of the eye, such as retinitis pigmentosa (RP) and glaucoma, by delivery of a transgene encoding a therapeutic polypeptide, such as glial cell-derived neurotrophic factor (GDNF), specifically to Müller glial cells using a gene delivery vector. In one embodiment, the gene delivery vector is a pseudotyped retroviral vector, particularly lentiviral vector.

Before the present invention is described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described. All publications mentioned herein are incorporated herein by reference to disclose and describe the methods and/or materials in connection with which the publications are cited. It is understood that the present disclosure supercedes any disclosure of an incorporated publication to the extent there is a contradiction.

It must be noted that as used herein and in the appended claims, the singular forms “a”, “an”, and “the” include plural referents unless the context clearly dictates otherwise. Thus, for example, reference to “a vector” includes a plurality of such vectors and reference to “the cell” includes reference to one or more cells and equivalents thereof known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### Definitions

“Gene” as used herein is meant to refer to at least a polynucleotide having at least a minimal sequence required for the expression of a coding sequence of interest. For example, “gene” minimally comprises a promoter that, when operably linked to a coding sequence of interest, facilitates expression of the coding sequence in a host cell. The coding sequence of the “gene” can be a genomic sequence (which includes one or more introns and exons) which, following splicing or rearrangement, provide for expression of a gene product of interest, or a recombinant polynucleotide, which lacks some or all intronic sequences (e.g., a cDNA).

The terms “polynucleotide” and “nucleic acid”, used interchangeably herein, refer to a polymeric forms of nucleotides of any length, either ribonucleotides or deoxynucleotides. Thus, these terms include, but are not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases. These comprise intronic and exonic sequences. In general, polynucleotides of interest in the present invention are those that are adapted for expression in a eukaryotic host cell, particularly a mammalian host cell, preferably a human cell, especially a cell of the eye (e.g., a retinal cell), particularly a mammalian (preferably human) cell of the eye.

The terms “polypeptide” and “protein”, used interchangeably herein, refer to a polymeric form of amino acids of any length, which in the context of the present invention, generally include amino acid residues that are genetically encodable. Polypeptides can also include those that are biochemically modified (e.g., post-translational modification such as glycosylation), as well as fusion proteins, including, but not limited to, fusion proteins with a heterologous amino acid sequence, fusions with heterologous and homologous leader sequences, with or without N-terminal methionine residues; immunologically tagged proteins; and the like.

The term “recombinant polynucleotide” as used herein intends a polynucleotide of genomic, cDNA, semisynthetic, or synthetic origin which, by virtue of its origin or manipulation: (1) is not associated with all or a portion of a polynucleotide with which it is associated in nature, (2) is linked to a polynucleotide other than that to which it is linked in nature, or (3) does not occur in nature.

“Operably linked” refers to a juxtaposition wherein the components so described are in a relationship permitting them to function in their intended manner. A control sequence “operably linked” to a coding sequence is ligated in such a way that expression of the coding sequence is achieved under conditions compatible with the control sequences.

An “open reading frame” (ORF) is a region of a polynucleotide sequence that encodes a polypeptide; this region may represent a portion of a coding sequence or a total coding sequence.

A “coding sequence” is a polynucleotide sequence that is transcribed into mRNA and/or translated into a polypeptide when placed under the control of appropriate regulatory sequences. The boundaries of the coding sequence are determined by a translation start codon at the 5′-terminus and a translation stop codon at the 3′-terminus. A coding sequence can include, but is not limited to mRNA, cDNA, and recombinant polynucleotide sequences.

“Transformation”, as used herein, refers to the insertion of an exogenous polynucleotide into a host cell, irrespective of the method used for the insertion, for example, viral infection, direct uptake, transduction, f-mating or electroporation. The exogenous polynucleotide may be maintained as a non-integrated vector, for example, an episomal element, or alternatively, may be integrated into the host genome.

“Subjects” or “patients” as used herein is meant to encompass any subject or patient amenable to application of the methods of the invention. Subjects include, without limitation, primate, canine, feline, bovine, equine, ovine, and avian subjects; mammals (particularly humans), domesticated pets (e.g., cat, dogs, birds, etc.) and livestock (cattle, swine, horses, etc.), and zoo animals being of particular interest.

The terms “treatment”, “treating”, “treat” and the like are used herein to generally refer to obtaining a desired pharmacologic and/or physiologic effect. The effect may be prophylactic in terms of completely or partially preventing a disease or symptom thereof and/or may be therapeutic in terms of a partial or complete stabilization or cure for a disease and/or adverse effect attributable to the disease. “Treatment” as used herein covers any treatment of a disease in a mammal, particularly a human, and includes: (a) preventing the disease or symptom from occurring in a subject which may be predisposed to the disease or symptom but has not yet been diagnosed as having it; (b) inhibiting the disease symptom, i.e., arresting its development; or (c) relieving the disease symptom, i.e., causing regression of the disease or symptom.

“Gene delivery vector” refers to a construct that is adapted for delivery of, and, within preferred embodiments facilitating expression, one or more gene(s) or sequence(s) of interest in a host cell. Representative examples of such vectors include viral vectors, nucleic acid expression vectors, naked DNA, and certain eukaryotic cells (e.g., producer cells).

“Diseases of the eye” or “eye condition” refers to a broad class of diseases or conditions wherein the functioning of the eye is affected due to damage or degeneration of the photoreceptors; or ganglia or optic nerve. Representative examples of such diseases include macular degeneration, diabetic retinopathies, inherited retinal degeneration such as retinitis pigmentosa, glaucoma, retinal detachment or injury and retinopathies (whether inherited, induced by surgery, trauma, a toxic compound or agent, or, photically).

The term “pseudotyped virion” as used herein, refers to a virion having an envelope protein that is not endogenous to the virion. Such pseudotyped virions can further be depleted for or lack the endogenous envelope protein, such that viral attachment is mediated by the non-endogenous viral envelope protein and will mediate fusion after interaction with its specific receptor. As fusion is determined by the envelope protein present at the surface of the virion, the fusion will occur and require the condition dictates by the envelope.

“Pseudotyping” as used herein refers to the ability of enveloped viruses such as lentiviruses to utilize envelope glycoproteins derived from other enveloped viruses. Pseudotyping, or the replacement of one virus's envelope glycoproteins with those from another virus, has been effective for increasing vector host cell range, increasing vector particle stability, and limiting vector entry to certain types of cells.

The term “producer cell” or “packaging cell” is used herein to refer to a host cell that supports production of viral particles according to the invention.

“Tropism” as used herein refers to the type of cell(s) that a particular vector prefers to transduce (enter) and express a gene product. The tropism of a vector may be altered by many factors including pseudotyping, transcriptional promoter elements, spatial and temporal delivery parameters, and species variability.

“Lentivector” or “lentivirus vector” or “LV” are used herein interchangeably to represent a recombinant self-inactivating replication incompetent viral vector with a genome based on a lentivirus (i.e. HIV-1). These vectors may have elements (i.e. envelope glycoproteins, enhancers, promoters) derived from other viruses including, but not limited to VSV, RRV, CMV, and hepatitis virus.

“Neurotrophic Factor” or “NT” as sued herein refers to proteins which are responsible for the development and maintenance of the nervous system. Representative examples of neurotrophic factors include GDNF, NGF, BDNF, CNTF, NT-3, NT-4, and Fibroblast Growth Factors.

### Overview

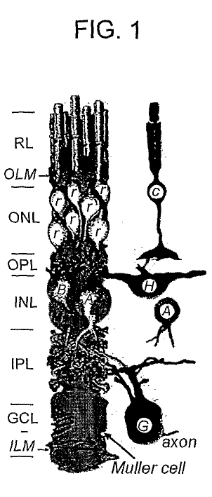

The present invention is based on the observation that it would be highly advantageous to deliver neuroprotective genes to Müller cells for retinal gene therapy. Müller cells are the most numerous glial cells in the eye (Liang et al. Adv Exp Med Biol 533, 439-45 2003), and can therefore serve as effective “bioreactors” for the secretion of neuroprotective factors. Additionally, Müller cells form a tight anatomical association with all other classes of retinal neurons that are affected by degenerative diseases (i.e. photoreceptors) (FIG. 1). Reports specify a Müller cell to cone photoreceptor ratio of 2:3 (Reichenbach et al. J Comp Neurol. 18; 360(2):257-70 1995, and Burris et al. J Comp Neurol. 453:100-111 2002) indicating that their numbers and anatomical association could provide an effective reservoir for neuroprotective factor secretion and disease therapy.

Moreover, Müller cells are accessible from the vitreous but span the entire retinal layer, all the way into the photoreceptor layer, and thus transducing this single cell type by intravitreal vector injection has the potential to mediate protection of the entire retina. Intravitreal injection is significantly less invasive and disruptive as compared to subretinal injection, and subretinal injection potentially only transduces a fraction of the retina. Furthermore, a natural function of Müller cells appears to be neuroprotection, particularly of photoreceptors (Wahlin et al. Invest Opthalmol Vis Sci 41, 927-36 2000 and Zack, Neuron 26, 285-6 2000). Therefore, gene delivery may be an effective approach to further exploit and enhance a natural role of these cells. Finally, it would likely be more advantageous to transduce Müller cells for indirect neuroprotection rather than the damaged or dying neurons themselves. None of the 150 retinitis pigmentosa (RP) associated mutations to date are Müller specific genes, indicating that these cells are potentially healthy, and therefore capable delivery targets, in at least some retinal disorders. However, there is unfortunately no effective vector system currently capable of efficient gene delivery to Müller cells.

The present invention thus concerns, in a general and overall sense, improved vectors that are designed to permit the transfection and transduction of retinal Müller glial cells, and provide high level expression of desired transgenes in such cells. Additionally, the present invention provides for restricted expression of these desired transgenes in that expression is regulated to achieve expression in specific cells.

The vectors of the present invention provide, for the first time, an efficient means of achieving cell type specific and high level expression of desired transgenes in retinal Müller glial cells. Müller glial cells have been difficult to transduce most probably because wild type viruses have evolved mechanisms to preferentially transduce neurons rather than glia, making Müller cells resistant to transduction by previous vector systems including Adenovirus, Adeno-associated virus, and Lentiviral vectors. The vectors of the present invention have the ability to infect non-dividing cells owing to the karyophilic properties of their preintegration complex, which allow for its active import through the nucleopore. Moreover, representative vectors of the present invention can mediate the efficient delivery, integration and appropriate or long-term expression of transgenes into non-mitotic cells both in vitro and in vivo. Müller cells transduced by the exemplary vectors of the present invention are capable of long-term expression. Most notably, however, the exemplary vectors of the present invention have highly desirable features that permit high level and specific expression of transgenes in Müller cells of the retina including mature, differentiated cells, while meeting human biosafety requirements.

The invention will now be described in more detail.

### Gene Delivery Vectors

Any of a variety of vectors adapted for expression of a therapeutic polypeptide in a cell of the eye, particularly within a Müller glial cell, are within the scope of the present invention. Gene delivery vectors can be viral (e.g., derived from or containing sequences of viral DNA or RNA, preferably packaged within a viral particle), or non-viral (e.g., not packaged within a viral particle, including “naked” polynucleotides, nucleic acid associated with a carrier particle such as a liposome or targeting molecule, and the like).

A particularly preferred gene delivery vector is a retroviral gene delivery vectors constructed to carry or express a selected gene(s) or sequence(s) of interest. Briefly, retroviral gene delivery vectors of the present invention may be readily constructed from a wide variety of retroviruses, including for example, B, C, and D type retroviruses as well as spumaviruses and lentiviruses (see RNA Tumor Viruses, Second Edition, Cold Spring Harbor Laboratory, 1985). Such retroviruses may be readily obtained from depositories or collections such as the American Type Culture Collection (“ATCC”; Rockville, Md.), or isolated from known sources using commonly available techniques.

Any of the above retroviruses may be readily utilized in order to assemble or construct retroviral gene delivery vectors given the disclosure provided herein, and standard recombinant techniques (e.g., Sambrook et al., Molecular Cloning: A Laboratory Manual, 2d ed., Cold Spring Harbor Laboratory Press, 1989; Kunkle, PNAS 82:488, 1985). In addition, within certain embodiments of the invention, portions of the retroviral gene delivery vectors may be derived from different retroviruses. For example, within one embodiment of the invention, retrovector LTRs may be derived from a Murine Sarcoma Virus, a tRNA binding site from a Rous Sarcoma Virus, a packaging signal from a Murine Leukemia Virus, and an origin of second strand synthesis from an Avian Leukosis Virus.

In some embodiments, the viral vectors of the present invention, therefore, may be generally described as recombinant vectors that include at least lentiviral gag, pol and rev genes, or those genes required for virus production, which permit the manufacture of vector in reasonable quantities using available producer cell lines. To meet important human safety needs, the more preferred vectors in accordance with the present invention will not include any other active lentiviral genes, such as vpr, vif, vpu, nef, tat. These genes may have been removed or otherwise inactivated. It is preferred that the only active lentiviral genes present in the vector will be the aforementioned gag, pol and rev genes.

A representative combination of lentiviral genes and backbone (i.e., long terminal repeats or LTRs) used in preparing lentivectors in accordance with the present invention will be one that is human immunodeficiency virus (HIV) derived, and more particularly, HIV-1 derived. Thus, the gag, pol, and rev genes will preferably be HIV genes and more preferably HIV-1 genes. However, the gag, pol, and rev genes and LTR regions from other lentiviruses may be employed for certain applications in accordance with the present invention, including the genes and LTRs of HIV-2, simian immunodeficiency virus (SIV), feline immunodeficiency virus, bovine immunodeficiency virus, equine infectious anemia virus, caprine arthritis encephalitis virus and the like. Such constructs could be useful, for example, where one desires to modify certain cells of non-human origin. However, the HIV based vector backbones (i.e. HIV LTR and HIV gag, pol and rev genes) will generally be preferred in connection with most aspects of the present invention in that HIV-based constructs are the most efficient at transduction of glial cells.

Other retroviral gene delivery vectors may likewise be utilized within the context of the present invention, including for example EP 0,415,731; WO 90/07936; WO 91/0285, WO 9403622; WO 9325698; WO 9325234; U.S. Pat. No. 5,219,740; WO 9311230; WO 9310218; Vile and Hart, Cancer Res. 53:3860-3864, 1993; Vile and Hart, Cancer Res. 53:962-967, 1993; Ram et al., Cancer Res. 53:83-88, 1993; Takamiya et al., J. Neurosci. Res. 33:493-503, 1992; Baba et al., J. Neurosurg. 79:729-735, 1993 (U.S. Pat. No. 4,777,127, GB 2,200,651, EP 0,345,242 and WO91/02805).

Packaging cell lines suitable for use with the above described retrovector constructs may be readily prepared (see U.S. Ser. No. 08/240,030, filed May 9, 1994, see also U.S. Ser. No. 07/800,921, filed Nov. 27, 1991), and utilized to create producer cell lines (also termed vector cell lines or “VCLs”) for the production of recombinant vector particles.

The viral vectors of the present invention also include an expression cassette comprising a transgene positioned under the control of a promoter that is active to promote detectable transcription of the transgene in a retinal cell. In preferred embodiments the promoter is active in promoting transcription of the transgene in Müller glial cells. Some embodiments include promoters that are active to promote transcription in specific cell types.

Examples of promoters suitable for use in connection with the present invention include a glial fibrillary acidic protein (GFAP), vimentin, glutamine synthetase, CD44, CRALBP, ubiquitin-C, CMV, CMV-beta-actin, PGK, and the EF1-alpha promoter. Of these, the GFAP promoter is particularly preferred. The GFAP promoter is an example of a promoter that provides for injury and stress regulated, specific expression restricted to desired cell types in that it promotes expression of the transgene primarily in glia. However, practice of the present invention is not restricted to the foregoing promoters, so long as the promoter is active in the glial cells.

To determine whether a particular promoter is useful, a selected promoter is tested in the construct in vitro in a Müller cell line and, if the promoter is capable of promoting expression of the transgene at a detectable signal-to-noise ratio, it will generally be useful in accordance with the present invention. Additionally, promoters deemed useful in vitro will be tested in vivo by methods of DNA electroporation to the retina. A desirable signal-to-noise ratio is one between about 10 and about 200, a more desirable signal-to-noise ratio is one 40 and about 200, and an even more desirable signal-to-noise ratio is one between about 150 and about 200. One means of testing such a promoter, described in more detail herein below, is through the use of a signal generating transgene such as the green fluorescent protein (GFP).

The present invention further provides for increased transduction efficiency through the inclusion of a central polypurine tract (cPPT) in the vector. The transduction efficiency may be 20%, 30%, 40%, 50%, 60%, 70%, or up to and including 80% transduction. In a preferred embodiment, the cPPT is positioned upstream of the promoter of sequence.

For certain applications, for example, in the case of promoters that are only modestly active in cells targeted for transduction, one will desire to employ a posttranscriptional regulatory sequence positioned to promote the expression of the transgene. One type of posttranscriptional regulatory sequence is an intron positioned within the expression cassette, which may serve to stimulate gene expression. However, introns placed in such a manner may expose the lentiviral RNA transcript to the normal cellular splicing and processing mechanisms. Thus, in particular embodiments it may be desirable to locate intron-containing transgenes in an orientation opposite to that of the vector genomic transcript.

A exemplary method of enhancing transgene expression is through the use of a posttranscriptional regulatory element which does not rely on splicing events, such as the posttranscriptional processing element of herpes simplex virus, the posttranscriptional regulatory element of the hepatitis B virus (HPRE) or that of the woodchuck hepatitis virus (WPRE), which contains an additional cis-acting element not found in the HPRE. The regulatory element is positioned within the vector so as to be included in the RNA transcript of the transgene, but outside of stop codon of the transgene translational unit. It has been found that the use of such regulatory elements is particularly preferred in the context of modest promoters, but may be contraindicated in the case of very highly efficient promoters.

In some embodiments the lentivectors of the present invention have an LTR region that has reduced promoter activity relative to wild-type LTR, in that such constructs provide a “self-inactivating” (SIN) biosafety feature. Self-inactivating vectors are ones in which the production of full-length vector RNA in transduced cells in greatly reduced or abolished-altogether. This feature greatly minimizes the risk that replication-competent recombinants (RCRs) will emerge. Furthermore, it reduces the risk that that cellular coding sequences located adjacent to the vector integration site will be aberrantly expressed. Furthermore, a SIN design reduces the possibility of interference between the LTR and the promoter that is driving the expression of the transgene. It is therefore particularly suitable to reveal the full potential of the internal promoter.

Self-inactivation may be achieved through the introduction of a deletion in the U3 region of the 3′ LTR of the vector DNA, i.e., the DNA used to produce the vector RNA. Thus, during reverse transcription, this deletion is transferred to the 5′ LTR of the proviral DNA. It is desirable to eliminate enough of the U3 sequence to greatly diminish or abolish altogether the transcriptional activity of the LTR, thereby greatly diminishing or abolishing the production of full-length vector RNA in transduced cells. However, it is generally desirable to retain those elements of the LTR that are involved in polyadenylation of the viral RNA, a function spread out over U3, R and U5. Accordingly, it is desirable to eliminate as many of the transcriptionally important motifs from the LTR as possible while sparing the polyadenylation determinants. In the case of HIV based lentivectors, it has been discovered that such vectors tolerate significant U3 deletions, including the removal of the LTR TATA box (e.g., deletions from −418 to −18), without significant reductions in vector titers. These deletions render the LTR region substantially transcriptionally inactive in that the transcriptional ability of the LTR in reduced to about 90% or lower. In preferred embodiments the LTR transcription is reduced to about 95% to 99%. Thus, the LTR may be rendered about 90%, 91%, 92%, 93%, 94%, 95% 96% 97%, 98%, to about 99% transcriptionally inactive.

The present invention describes gene transfer vehicles that appear particularly well suited for the transduction of retinal Müller glial cells and for the expression of transgenes under the control of specific transcription factors. These vectors will facilitate the further use of lentiviral vectors for the genetic manipulation of Müller glial cells, and should be particularly useful for both research and therapeutic applications.

### Conditions Amenable to Treatment

The methods of the invention can be used to treat (e.g., prior to or after the onset of symptoms) in a susceptible subject or subject diagnosed with a variety of eye diseases. The eye disease may be a results of environmental (e.g., chemical insult, thermal insult, and the like), mechanical insult (e.g., injury due to accident or surgery), or genetic factors. The subject having the condition may have one or both eyes affected, and therapy may be administered according to the invention to the affected eye or to an eye at risk of disease, such as photoreceptor degeneration, due to the presence of such a condition in the subject's other, affected eye.

The present invention provides methods which generally comprise the step of intraocularly administering (e.g., by subretinal injection) a gene delivery vector which directs the expression of a therapeutic polypeptide, such as the neurotrophic factor GDNF, to the eye to treat, prevent, or inhibit the progression of an eye disease. As utilized herein, it should be understood that the terms “treated, prevented, or, inhibited” refers to the alteration of a disease onset, course, or progress in a statistically significant manner.

Another condition amenable to treatment according to the invention is Age-related Macular Degeneration (AMD). The macula is a structure near the center of the retina that contains the fovea. This specialized portion of the retina is responsible for the high-resolution vision that permits activities such as reading. The loss of central vision in AMD is devastating. Degenerative changes to the macula (maculopathy) can occur at almost any time in life but are much more prevalent with advancing age. Conventional treatments are short-lived, due to recurrent choroidal neovascularization. AMD has two primary pathologic processes, choroidal neovascularization (CNV) and macular photoreceptor cell death. Delivery of GDNF to the eye according to the present invention can ameliorate the photoreceptor cell death. Administration of GDNF has a distinct advantage relative to other NTFs (such as FGF-2) in that GDNF is not angiogenic. Thus GDNF may be the NTF of choice to treat AMD to preserve macular cones without exacerbating the CNV.

As noted above, the present invention also provides methods of treating, preventing, or, inhibiting neovascular disease of the eye, comprising the step of administering to a patient a gene delivery vector which directs the expression of an anti-angiogenic factor. Representative examples of neovascular diseases include diabetic retinopathy, ARMD (wet form), and retinopathy of prematurity. Briefly, choroidal neovascularization is a hallmark of exudative or wet Age-related Macular Degeneration (AMD), the leading cause of blindness in the elderly population. Retinal neovascularization occurs in diseases such as diabetic retinapathy and retinopathy of prematurity (ROP), the most common cause of blindness in the young. In such embodiments, suitable vectors for the treatment, prevention, or, inhibition of neovascular diseases of the eye direct the expression of an anti-angiogenic factor such as, for example, soluble tie-2 receptor or soluble Flt-1.

Exemplary conditions of particular interest which are amenable to treatment according to the methods of the invention include, but are not necessarily limited to, retinitis pigmentosa (RP), diabetic retinopathy, and glaucoma, including open-angle glaucoma (e.g., primary open-angle glaucoma), angle-closure glaucoma, and secondary glaucomas (e.g., pigmentary glaucoma, pseudoexfoliative glaucoma, and glaucomas resulting from trauma and inflammatory diseases).

In one embodiment of this invention may be the secretion of a rod derived cone, survivability factor (rdCVF) (Leveillard et al. Nat Genet. 2004 July; 36(7):755-9) from healthy Müller cells for the protection of diseased and degenerate cone photoreceptors in the retina. This could have significant relevance to the treatment of AMD and RP.

Further exemplary conditions amenable to treatment according to the invention include, but are not necessarily limited to, retinal detachment, age-related or other maculopathies, photic retinopathies, surgery-induced retinopathies, toxic retinopathies, retinopathy of prematurity, retinopathies due to trauma or penetrating lesions of the eye, inherited retinal degenerations, surgery-induced retinopathies, toxic retinopathies, retinopathies due to trauma or penetrating lesions of the eye.

Specific exemplary inherited conditions of interest for treatment according to the invention include, but are not necessarily limited to, Bardet-Biedl syndrome (autosomal recessive); Congenital amaurosis (autosomal recessive); Cone or cone-rod dystrophy (autosomal dominant and X-linked forms); Congenital stationary night blindness (autosomal dominant, autosomal recessive and X-linked forms); Macular degeneration (autosomal dominant and autosomal recessive forms); Optic atrophy, autosomal dominant and X-linked forms); Retinitis pigmentosa (autosomal dominant, autosomal recessive and X-linked forms); Syndromic or systemic retinopathy (autosomal dominant, autosomal recessive and X-linked forms); and Usher syndrome (autosomal recessive).

Assessment of Treatment

The effects of therapy according to the invention as described herein can be assessed in a variety of ways, using methods known in the art. For example, the subjects vision can be tested according to conventional methods. Such conventional methods include, but are not necessarily limited to, electroretinogram (ERG), focal ERG, tests for visual fields, tests for visual acuity, ocular coherence tomography (OCT), Fundus photography, Visual Evoked Potentials (VEP) and Pupillometry. In general, the invention provides for maintenance of a subject's vision (e.g., prevention or inhibition of vision loss of further vision loss due to photoreceptor degeneration), slows progression of vision loss, or in some embodiments, provides for improved vision relative to the subject's vision prior to therapy.

### Methods of Administration

The gene delivery vectors of the present invention can be delivered to the eye through a variety of routes. They may be delivered intraocularly, by topical application to the eye or by intraocular injection into, for example the vitreous or subretinal (interphotoreceptor) space. Alternatively, they may be delivered locally by insertion or injection into the tissue surrounding the eye. They may be delivered systemically through an oral route or by subcutaneous, intravenous or intramuscular injection. Alternatively, they may be delivered by means of a catheter or by means of an implant, wherein such an implant is made of a porous, non-porous or gelatinous material, including membranes such as silastic membranes or fibers, biodegradable polymers, or proteinaceous material. The gene delivery vector can be administered prior to the onset of the condition, to prevent its occurrence, for example, during surgery on the eye, or immediately after the onset of the pathological condition or during the occurrence of an acute or protracted condition.

The gene delivery vector can be modified to enhance penetration of the blood-retinal barrier. Such modifications may include increasing the lipophilicity of the pharmaceutical formulation in which the gene delivery vector is provided.

The gene delivery vector can be delivered alone or in combination, and may be delivered along with a pharmaceutically acceptable vehicle. Ideally, such a vehicle would enhance the stability and/or delivery properties. The invention also provides for pharmaceutical compositions containing the active factor or fragment or derivative thereof, which can be administered using a suitable vehicle such as liposomes, microparticles or microcapsules. In various embodiments of the invention, it may be useful to use such compositions to achieve sustained release of the active component.

The amount of gene delivery vector (e.g., the number of viral particles), and the amount of the therapeutic polypeptide expressed, effective in the treatment of a particular disorder or condition will depend of the nature of the disorder or condition and a variety of patient-specific factors, and can be determined by standard clinical techniques.

In a representative embodiment, the gene delivery vectors are administered to the eye, such as intraocularly to a variety of locations within the eye depending on the type of disease to be treated, prevented, or, inhibited, and the extent of disease. Examples of suitable locations include the retina (e.g., for retinal diseases), the vitreous, or other locations in or adjacent the retina or in or adjacent the eye.

Briefly, the human retina is organized in a fairly exact mosaic. In the fovea, the mosaic is a hexagonal packing of cones. Outside the fovea, the rods break up the close hexagonal packing of the cones but still allow an organized architecture with cones rather evenly spaced surrounded by rings of rods. Thus in terms of densities of the different photoreceptor populations in the human retina, it is clear that the cone density is highest in the foveal pit and falls rapidly outside the fovea to a fairly even density into the peripheral retina (see Osterberg, G. (1935) Topography of the layer of rods and cones in the human retina. Acta Ophthal. (suppl.) 6, 1-103; see also Curcio, C. A., Sloan, K. R., Packer, O., Hendrickson, A. E. and Kalina, R. E. (1987) Distribution of cones in human and monkey retina: individual variability and radial asymmetry. Science 236, 579-582).

Access to desired portions of the retina, or to other parts of the eye may be readily accomplished by one of skill in the art (see, generally Medical and Surgical Retina: Advances, Controversies, and Management, Hilel Lewis, Stephen J. Ryan, Eds., medical—” illustrator, Timothy C. Hengst. St. Louis: Mosby, c1994. xix, 534; see also Retina, Stephen J. Ryan, editor in chief, 2nd ed., St. Louis, Mo.: Mosby, c1994. 3 v. (xxix, 2559 p.).

The amount of the specific viral vector applied to the retina is uniformly quite small as the eye is a relatively contained structure and the agent is injected directly into it. The amount of vector that needs to be injected is determined by the intraocular location of the chosen cells targeted for treatment. The cell type to be transduced will be determined by the particular disease entity that is to be treated.

For example, a single 20 μl volume (of 10⁹ transducing units/ml LV) may be used in a subretinal injection to treat the macula and fovea. A larger injection of 50 μl to 100 μl may be used to deliver the LV to a substantial fraction of the retinal area, perhaps to the entire retina depending upon the extent of lateral spread of the particles. A 100 μl injection will provide several million active LV particles in to the subretinal space. This calculation is based upon a titer of 10⁹ infectious particles per milliliter. The retinal anatomy constrains the injection volume possible in the subretinal space (SRS). Assuming an injection maximum of 100 μL, this would provide an infectious titer of 10⁸ LV in the SRS. This would have the potential of infecting a large majority of the Müller cells in the entire human retina with a single injection.

Smaller injection volumes focally applied to the fovea or macula may adequately transfect the entire region affected by the disease in the case of macular degeneration or other regional retinopathies.

Gene delivery vectors can alternately be delivered to the eye by intraocular injection into the vitreous. In this application, the primary target cells to be transduced are Müller cells and retinal ganglion cells, the former being the retinal cells primarily affected in glaucoma. In this application, the injection volume of the gene delivery vector could be substantially larger, as the volume is not constrained by the anatomy of the interphotoreceptor or subretinal space. Acceptable dosages in this instance can range from 25 μl to 1000 μl.

### Pharmaceutical Compositions

Gene delivery vectors can be prepared as a pharmaceutically acceptable composition suitable for administration. In general, such pharmaceutical compositions comprise an amount of a gene delivery vector suitable for delivery of transgene encoding a therapeutic polynucleotide, such as GDNF, to the Müller glial cells of the eye for expression of a therapeutically effective amount of the polypeptide, combined with a pharmaceutically acceptable carrier or excipient. Preferably, the pharmaceutically acceptable carrier is suitable for intraocular administration. Exemplary pharmaceutically acceptable carriers include, but are not necessarily limited to, saline or a buffered saline solution (e.g., phosphate-buffered saline).

Various pharmaceutically acceptable excipients are well known in the art. As used herein, “pharmaceutically acceptable excipient” includes any material which, when combined with an active ingredient of a composition, allows the ingredient to retain biological activity, preferably without causing disruptive reactions with the subject's immune system or adversely affecting the tissues surrounding the site of administration (e.g., within the eye).

Exemplary pharmaceutically carriers include sterile aqueous of non-aqueous solutions, suspensions, and emulsions. Examples include, but are not limited to, any of the standard pharmaceutical excipients such as a saline, buffered saline (e.g., phosphate buffered saline), water, emulsions such as oil/water emulsion, and various types of wetting agents. Examples of non-aqueous solvents are propylene glycol, polyethylene glycol, hyaluronic acid, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's or fixed oils. Intravenous vehicles can include fluid and nutrient replenishers, electrolyte replenishers (such as those based on Ringer's dextrose), and the like.

A composition of gene delivery vector of the invention may also be lyophilized using means well known in the art, for subsequent reconstitution and use according to the invention. Where the vector is to be delivered without being encapsulated in a viral particle (e.g., as “naked” polynucleotide), formulations for liposomal delivery, and formulations comprising microencapsulated polynucleotides, may also be of interest.

Compositions comprising excipients are formulated by well known conventional methods (see, for example, Remington's Pharmaceutical Sciences, Chapter 43, 14th Ed., Mack Publishing Col, Easton Pa. 18042, USA).

In general, the pharmaceutical compositions can be prepared in various forms, preferably a form compatible with intraocular administration. Stabilizing agents, wetting and emulsifying agents, salts for varying the osmotic pressure or buffers for securing an adequate pH value may also optionally be present in the pharmaceutical composition.

The amount of gene delivery vector in the pharmaceutical formulations can vary widely, i.e., from less than about 0.1%, usually at or at least about 2% to as much as 20% to 50% or more by weight, and will be selected primarily by fluid volumes, viscosities, etc., in accordance with the particular mode of administration selected.

The pharmaceutical composition can comprise other agents suitable for administration, which agents may have similar to additional pharmacological activities to the therapeutic protein to be delivered (e.g., GDNF).

### Kits

The invention also provides kits comprising various materials for carrying out the methods of the invention. In one embodiment, the kit comprises a vector encoding a GDNF polypeptide, which vector is adapted for delivery to a subject, particularly the Müller glial cells of the subject, and adapted to provide for expression of the therapeutic polypeptide in the Müller glial cells of an eye. The kit can comprise the vector in a sterile vial, which may be labeled for use. The vector can be provided in a pharmaceutical composition. In one embodiment, the vector is packaged in a virus. The kit can further comprise a needle and/or syringe suitable for use with the vial or, alternatively, containing the vector, which needle and/or syringe are preferably sterile. In another embodiment, the kit comprises a catheter suitable for delivery of a vector to the eye, which catheter may be optionally attached to a syringe for delivery of the vector. The kits can further comprise instructions for use, e.g., instructions regarding route of administration, dose, dosage regimen, site of administration, and the like.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Methods and Materials

The following methods and materials were used in the examples below.

### LV Transfer Vector Design, Promoter Selection, and Construction

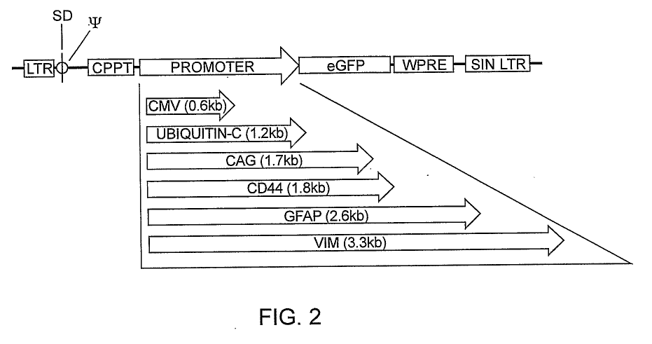

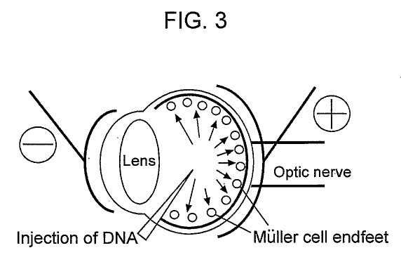

Prior to the construction of LV vectors, appropriate glial specific promoter elements were selected to drive GFP expression in Müller cells. GFP expression constructs containing candidate Müller cell specific promoters (i.e. human GFAP, mouse GFAP, mouse Vimentin, rat Glutamine Synthetase, mouse CD44, human CRALBP) were prepared and electroporated into retinas of young wild type rodents as described in Matsuda and Cepko, PNAS, 2003 (FIG. 2). Briefly, two sets of five square wave pulses (100 mA pulses, 100 ms duration, 1 Hz) separated by a five minute rest were applied to the eye following subretinal or intravitreal injection of naked plasmid DNA (FIG. 3). Expression was then evaluated by fundus photography or cryosections 2-3 days following injection. Promoters that exhibited high levels of Müller specific expression were selected for use in the LV vector experiments.

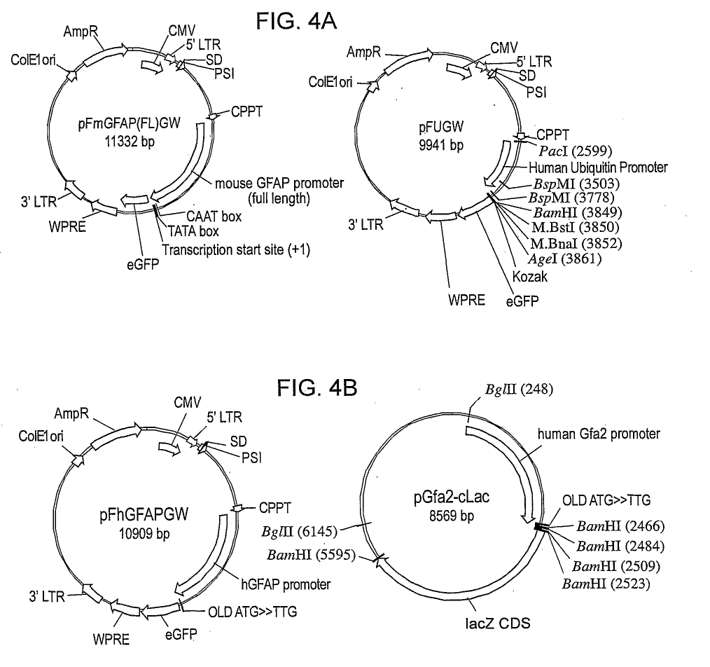

HIV-1 based transfer vector plasmids were derived from the plasmids pCS-CG or pFUGW. Vectors containing both the CPPT and WPRE elements were based on pFUGW. To start, the mouse glial fibrillary acidic protein (mGFAP) full length (FL) promoter was high fidelity PCR amplified from genomic mouse tail DNA using the primers (Forward: 5′-CCGCGGAAAGCTTAGACCCAAG-3′ (SEQ ID NO:01) and Reverse: 5′-GCTAGCTTCCTGCCCTGCCTCT-3′ (SEQ ID NO:02)). To construct pFmGFAP(FL)GW the 2.6 kb GFAP promoter fragment was subcloned to replace the Ubiquitin-C promoter in pFUGW (FIG. 4A). For human GFAP promoted vectors, the human GFAP promoter was released from pGfa2-cLac by BglII-BamHI digest, blunted and ligated in place of the Ubiquitin-C promoter in pFUGW to create pFhGFAPGW (FIG. 4B).

### Vector Production

All procedures involving vector production, concentration, and titration were performed in a Type IIA biosafety cabinet under strict BL2 practice. LV vectors were produced by either calcium phosphate or Lipofectamine 2000 (Invitrogen) transient transfection.

Calcium phosphate transfections were performed as follows. Five T-175 (Nunc) flasks were coated with poly-L-lysine (Sigma #P4832 diluted 1:10 in PBS and sterile filtered) and allowed to stand for 10 minutes before aspirating. Low passage 293T cells (ATCC #CRL-11268) were seeded at 1.2-1.5×10⁷ cells per flask in 20 mL complete IMDM (IMDM+10% FBS, 1×Pen/Strep, 2 mM L-glutamine). The following day the calcium-phosphate/DNA precipitate was prepared after all reagents equilibrated to room temperature. For five T-175 flasks, 158 μg transfer vector (i.e. pFmGFAP(FL)GW or pFhGFAPGW), 79 μg pMDLg/pRRE, 24 μg pRSV-REV, and 55 kg envelope glycoprotein (i.e. RRV or VSVG) were mixed in a final volume of 13.9 mL sterile ddH₂0 (buffered with Hepes to 2.5 mM) and 1.9 mL 2.5M CaCl₂. After mixing, 15.8 mL 2×HeBS (Hepes Buffered Saline pH 7.05) was added to the DNA/H₂O/CaCl₂ solution and mixed by pipetting briefly. The CaPO₄ precipitate formed during a 1.5 minute incubation, and the reaction was quenched by adding 18.4 mL complete. IMDM media. After mixing briefly, 10 mL of this solution was added to each flask which was placed in an incubator (37° C., 5% CO₂) overnight. Media was aspirated and replaced with 20 mL fresh IMDM 12 hours later. Two harvests of the cell supernatant were performed 24 hours and 48 hours after the first media change. The cell supernatant (200 mL) was then filtered through a 0.45 μm pore PVDF Durapore filter (Millipore, Bedford, Mass.) and stored at 4° C. until concentrated.

For Lipofectamine 2000 transfections, 293T cells were plated in complete IMDM lacking antibiotics. For optimal transfections, the total amount of plasmid DNA was reduced by 2.25 fold, while maintaining the above ratio of four plasmids. Transfection complexes were prepared by mixing the plasmids in a final volume of 21.9 mL Opti-MEM reduced serum media (Invitrogen). In a separate reaction tube, 21.4 mL Opti-MEM media was gently mixed with 525 μL Lipofectamine 2000 reagent. Both tubes were incubated at room temperature for 5 minutes, gently mixed together, and incubated another 20 minutes. This solution was added to each of the five flasks which were placed in an incubator overnight. Transfection media was aspirated 12 hours later, cells were washed with PBS, and given 20 mL complete IMDM. The additional PBS wash was found necessary to remove transfection amine complexes which frequently caused cataracts when carried over into the injected vector preparation. Vector supernatant was harvested and filtered as described above.

### Vector Concentration for In Vivo Use

High titer LV vector stocks were generated after two rounds of ultracentrifugation. The filtered vector supernatant (32 mL) was carefully overlaid on a 20% sucrose solution (4 mL) in six ultracentrifuge tubes (Beckman #344058) which were centrifuged at 24,000 rpm in a SW-28 rotor for 2 hours at 4° C. The supernatant was aspirated (avoiding the pellet) and 800 μL cold PBS was added to each tube and mixed by pipetting. After a 30 minute incubation on ice, the six vector/PBS tubes were pooled and overlayed on 1 mL of 20% sucrose in one ultracentrifuge tube (Beckman #344059). The vector was centrifuged in a SW-41Ti rotor at 25,000 rpm for 1.5 hours at 4° C. The supernatant was aspirated and pelleted vector was resuspended in 200 μL cold PBS. Vector was incubated on ice overnight and again mixed by pipetting. If not used immediately, vector was stored for up to one week at 4° C. or flash frozen and stored at −80° C. for long term.

### Vector Titration by Q-PCR

Both physical particle and functional biological titers may be determined by several methods including p24 ELISA, FACS, and quantitative PCR. A particle titer estimates the amount of vector present in a preparation, however it provides no information regarding the biological function of a vector. Conversely, functional titer determination can accurately estimate the infectious ability of a vector through the quantitative detection of integrated proviral genomes by real time PCR. This method has the advantage of isolating the viral transduction event from later gene transcription and translation, which is the basis for protein expression titers (FACS). Although time consuming, one clear benefit to this approach was the ability to determine vector titer on a cell line (i.e., 293s) irrespective of the vector delivered promoter element. Vectors may contain cell specific promoters whose gene product is not expressed in an available cell line, and therefore titer determination based on protein expression is not feasible. Additionally, this method was found to be invaluable for testing vector transduction efficiency of pseudotyped or engineered vectors on primary retinal cell isolates regardless of promoter.

Functional titer was determined based on the protocol (Sastry et al. Gene Therapy 9, 1155-1162, 2002) by quantitative PCR as follows. Cultured 293T cells were infected with serial dilutions of vector (10⁻³-10⁻⁷) in 1.0 mL media with 8 ug/mL polybrene in a six well plate (2-5×10⁵ cells/well). Cells were incubated for at least 4-5 days and washed multiple times to remove residual plasmid carried over from vector production. The transduced cells were then trypsinized, counted, and DNA from 1×10⁶ cells from each well was isolated (Gentra Puregene #D-5000A). The total amount of DNA from each sample was normalized and 5 was added to each Q-PCR reaction (ABI #N808-0228) containing 3.5 mM MgCl₂, 200 μM each DNTP, 320 nM each primer, 320 nM probe, 0.025 U/μL amplitaq, 2.5 μL reaction buffer, and ddH₂O to 25 μL. Primers (Forward: 5′-ACCTGAAAGCGAAAGGGAAAC-3′ (SEQ ID NO:03), Reverse: 5′-CACCCATCTCTCTCCTTCTAGCC-3′ (SEQ ID NO:04)) and probe (5′ FAM-AGCTCTCTCGACGCAGGACTC GGC-BHQ-3′ Biosearch Technologies (SEQ ID NO:05)) sequences specific to the HIV-1 packaging signal (ψ) were used with any HIV-1 based vectors containing this element. A standard curve was generated by amplification of a spectrophotometrically predetermined quantity (10¹⁰-10² molecules/reaction) of transfer vector plasmid containing the HIV-1 packaging sequence.

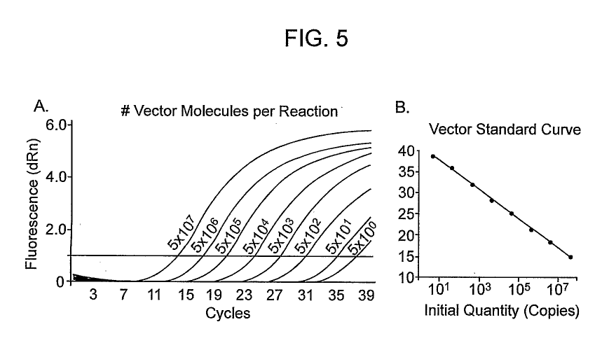

Each reaction was performed in triplicate under the following conditions in a Stratagene Mx-3000P thermocycler: 1 cycle of 95° C. for 10 minutes, 40 cycles of 95° C. for 15 seconds and 60° C. for 2 minutes. The thermocycler was set to detect and report fluorescence during the annealing/extension step of each cycle. A standard curve was generated by plotting threshold cycles vs. copy number and vector DNA titer in TU/mL (transducing units/mL) was determined at multiple dilutions (FIG. 5).

An RNA based particle titer was also determined using Quantitative Reverse Transcriptase PCR (QRT-PCR). Serial dilutions of vector stock were prepared in PBS, RNA was extracted (QIAamp MinElute Virus Kit Qiagen #57714), and residual DNA removed while RNA was bound to the purification column (Qiagen Rnase-Free Dnase set #79254). QRT-PCR reactions (Stratagene Brilliant QRT-PCR Master Mix Kit #600551) were prepared as follows: 1×QRT-PCR Master Mix, 320 nM each primer (see above), 320 nM probe (see above), 0.375 μL of 1:500 diluted reference dye, 0.1 μL StrataScript RT/Rnase, and ddH₂O to 25 μL. Reactions were performed in triplicate and reactions lacking RT were used to determine background DNA amplification. Cycling conditions were as described above with the addition of an initial 48° C. RT cycle for 30 minutes. RNA titer was determined by using transfer vector plasmid as the standard after subtracting out background signal from the reactions lacking RT.

DNA based functional vector titers in the cell supernatant ranged from 5×10⁶-2×10⁷ TU/mL before and 7×10⁸-1×10¹⁰ TU/mL after concentration. RNA based particle titers were 3×10⁸-8×10⁹ particles/mL in the supernatant, and 6×10¹⁰-2×10¹² particles/mL after concentration. Taking the difference between RNA and DNA titers, it was found that the functional vector:inactive particle ratio to be from 1:100 to 1:1000. GFP titers were also determined by direct visualization for some vector batches and were found to be slightly lower than Q-PCR determined functional titers. Titers of vectors produced by Lipofectamine 2000 transfection were routinely higher than those produced by calcium phosphate transfection.

### Intraocular Injection Procedure

All procedures used were in accordance with the ARVO Statement for the Use of Animals in Ophthalmic and Vision Research and were approved by the University of California, Berkeley Committee on Animal Research. Animals were anesthetized by intraperitoneal injection of ketamine/xylazine and eyes were dilated using 2.5% phenylephrine hydrochloride and 1% atropine sulfate. A shelving puncture was made through the sclera with a sharp 30-gauge needle, followed by a Hamilton syringe equipped with a blunt 33-gauge needle. For subretinal injections, the tip of the needle was advanced through the sclera, choroid, retina, and vitreous, and the needle penetrated the superior central retina to deliver the vector (0.5-3 μL) into the subretinal space. It was found this approach to be most successful in avoiding damage to the lens. Intravitreal injections were performed by delivering the vector (2-10 μL) directly into the vitreous body. Immediately after injection, the quality (i.e., lack of hemorrhage) and size of the subretinal bleb were evaluated under a stereo microscope by visualizing through a cover slip with Celluvisc (Allergan, Irvine, Calif.) placed on the cornea

### Tissue Preparation

Eyes were enucleated from animals injected with LV-mGFAP-GFP or LV-hGFAP-GFP at 10-60 days post-injection. Eye-cups were fixed in 4% paraformaldehyde in PBS for 1 hour at 4° C. and washed in PBS. Eyes were cryoprotected in 15% sucrose for 2 hours followed by 30% sucrose overnight at 4° C., embedded in OCT, and flash frozen in a dry ice/ethanol slurry. Sections were cut (10 μM thick) using a CM1850 cryostat (Leica, Nussloch, Germany) and were thaw mounted on Superfrost Plus slides (Fisher Scientific). Alternatively, eyes were briefly fixed, imbedded in 5% agarose and sectioned (100 μm thickness) on a Leica VT1000S vibratome. Images were acquired using a Zeiss Axiophot epifluorescence, Zeiss Axioplan 2e deconvolution, or Zeiss 510 META Axioplan2 confocal microscope (Thornwood, N.Y.).

### In Vivo Results

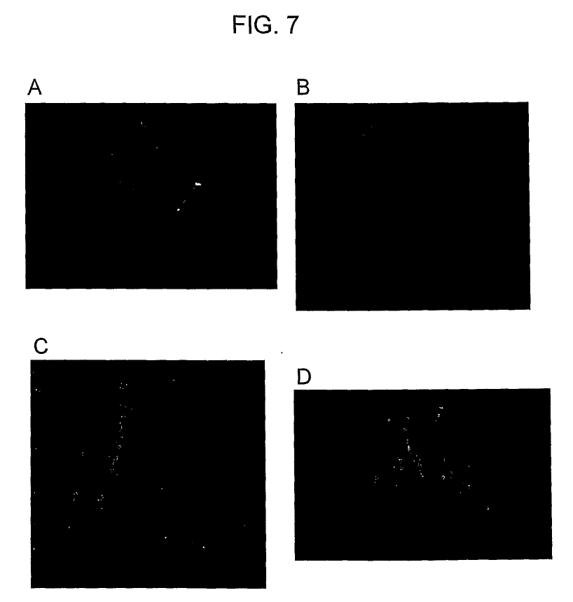

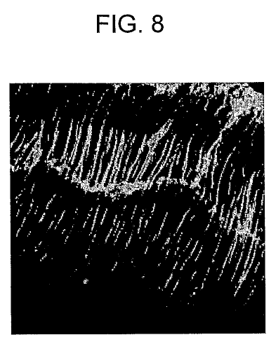

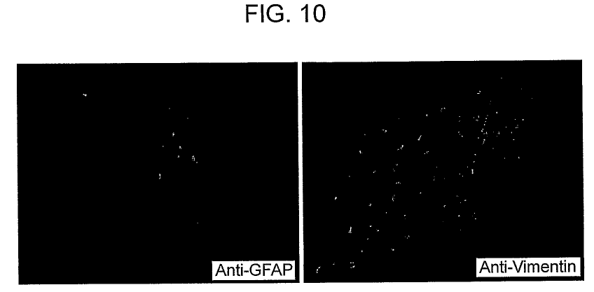

The LV vectors were delivered to rodents via intraocular injection as described. Following intravitreal injection the VSV-mGFAP-GFP LV vector successfully transduced Müller cells around the injection site (FIG. 8). When viewed in thick 100 μm section and imaged via deconvolution or confocal microscopy, the distinctive Müller cell anatomy is revealed showing strong GFP expression (FIG. 7, panels A-D). Retinas were also stained with α-vimentin antibody (FIG. 8) to label Müller cells and confirm anatomical Müller cell structures expressing LV delivered GFP. Fundus images reveal extent of GFP expression in the rat retina 10 days following subretinal injection of Müller specific vector (FIG. 10).

### In Vivo GFP Imaging

Fundus imaging was performed 2-180 days after injection of LV vectors using a Retcam II (Clarity Medical Systems Inc., Pleasanton, Calif.) equipped with a wide angle 130 degree Retinopathy of Prematurity (ROP) lens to monitor GFP expression in live anesthetized rats.

### Tissue Preparation

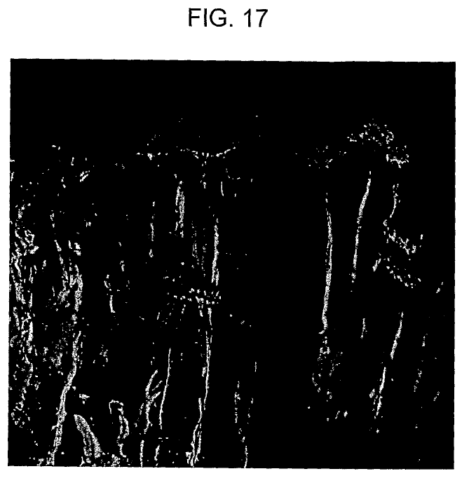

Retinas were detached from the RPE and fixed in 4% formaldehyde (1 hr), embedded in molten (42° C.) 5% agarose in PBS, and 100 μm thick sections were cut on a Leica VT1000S vibratome. For cryosections, eyes were fixed, cryoprotected in 15% followed by 30% sucrose, embedded in OCT Tissue-TEK (Sakura Finetek U.S.A. Inc., Torrance, Calif.) and sectioned at 16 μm using a Leica CM1850 cryostat. Immunohistochemistry was performed as described32 using α-GS (BD #610517, 1:1000) or α-Rhodopsin (Rho4D2, 1:100, gift of Robert Molday) primary antibodies, and detected using an Alexa Fluor 633 (Molecular Probes #A21052, 1:1000) secondary antibody. Serial confocal images were acquired on a Zeiss LSM-510 META confocal microscope (40× Plan Neofluar 1.3 N.A. or 63× Plan Apochromat 1.4 N.A. oil objectives). Full field 1024×1024 optical sections were made in 0.37 μm steps (146 sections for FIG. 17), and 3D reconstructions generated using Imaris software (Bitplane Inc., Saint Paul, Minn.).

### Electroretinograms

Rats were dark-adapted 12 hrs overnight, anesthetized, and eyes dilated. Contact lens electrodes were placed on each cornea and reference electrodes were placed subcutaneously under each eye. Light stimulus was presented in a series of seven flashes with increasing intensity from 0.0001-1.0 (cd-s)/m2, and responses were recorded using an Espion ERG system (Diagnosys LLC, Littleton, Mass.). A-wave amplitudes were measured from baseline to the corneal negative peak and b-wave amplitudes from the corneal negative peak to the major corneal positive peak after subtracting any contributions due to oscillatory potentials. Three responses were averaged for each light intensity.

### Transduction Area Measurements

Total retinal surface area expressing GFP after subretinal injection vector was determined from fluorescent fundus images. Surface area measurements were based upon a 3.39 mm radius eye having a total retinal surface area of 80.64 mm2 (56% of the entire sphere). 33 Fundus images were calibrated for scale by measuring retinal vessel diameters (44.2+3.8 nm) near the optic disc as seen in confocal images of flat mount retinal preparations with Zeiss LSM 5 software.

### Example 1

### In Vitro and In Vivo Characterization of RRV Pseudotyped LV Vectors

Lentiviral vector particles were constructed as described above to contain envelope glycoproteins (pseudotyped) derived from the Ross River Virus (RRV). RRV is an enveloped retrovirus that was first isolated from mosquitoes in the Ross River, Australia. It exhibits an extremely broad host range and RRV infection leads to epidemic polyarthritis in humans.

RRV pseudotyped LV vector particles were packaged as described above and concentrated to high titer (10⁸-10⁹ TU/mL). Titer was determined by Q-PCR and direct GFP visualization as described above.

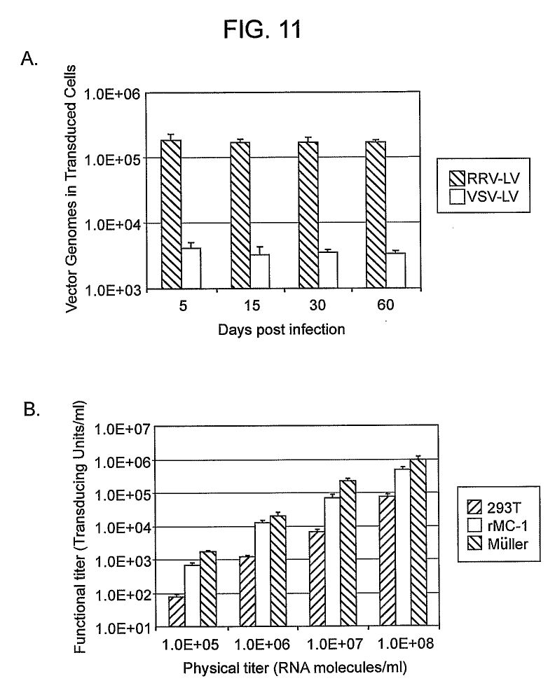

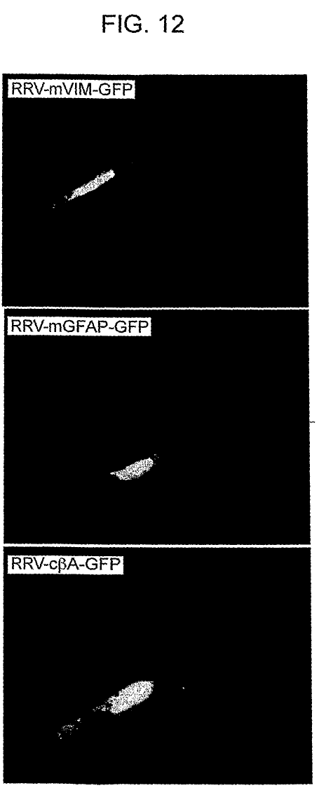

In vitro characterization was performed as follows. RRV-LV (CMV-GFP) vector particles were added to in vitro cultures of 293T, primary Müller, and a rat Müller cell line rMC-1 (Sarthy et al. IOVS V39 212-215 1998) along with polybrene (8 μg/mL). These Müller cell lines were stained with antibodies to GFAP and Vimentin and exhibit an expression profile similar to their in vivo counterpart (FIG. 10). Transduction efficiency was determined on these three cell lines based on DNA and RNA vector titers by Q-PCR as described above. Transduction of primary rat Müller cells by RRV-LV was 50 fold more efficient than VSV-LV (FIG. 12, panel A). RRV-LV transduces primary rat Müller cells 20 fold more efficiently and rMC-1 cells 9 fold more efficiently than HEK 293T cells (FIG. 11, panel B). Transduction is stable for at least 60 days in primary rat Müller cells. Although viral genomes were detectable in high levels by Q-PCR showing efficient cell entry, limited expression was observed with the CMV promoter.

To achieve in vitro expression in cultured Müller cells, multiple RRV pseudotyped LV vectors were constructed, packaged, and delivered as described above. RRV-LV vectors containing ubiquitous (CPA) and cell specific (GFAP and Vimentin) promoters successfully drove GFP expression in cultured Müller cells (FIG. 12).

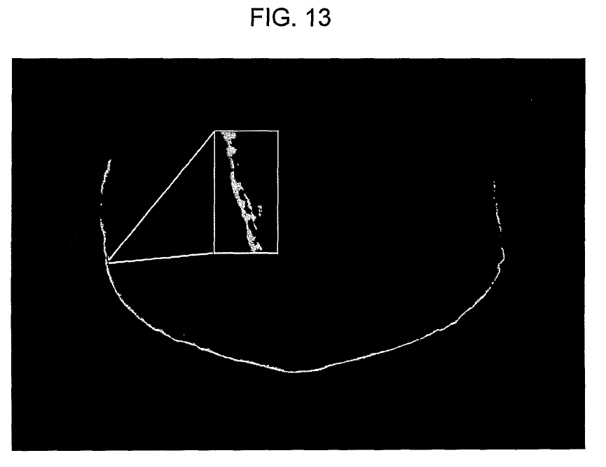

For in vivo studies, the above described vector (RRV-CMV-GFP) was administered via intravitreal or subretinal injection to rodent retinas. Limited expression was observed in lens epithelium following intravitreal injection. Following subretinal injection, strong expression was seen in the RPE covering the majority of the RPE layer (FIG. 13). As in the in vitro studies, no expression was seen in vivo in Miller cells with the CMV promoter. This lack of expression is likely due to the viral CMV promoter's preference to express in neurons and epithelium in the retina rather than glia. In vivo GFP expression with RRV-LV vectors delivered to the vitreous was observed when vectors contained Müller cell specific promoters such as GFAP.

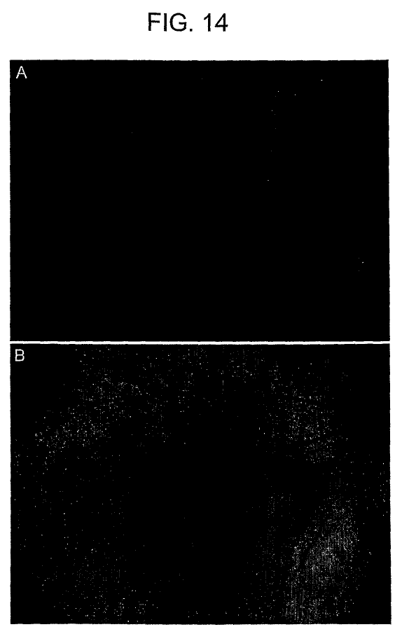

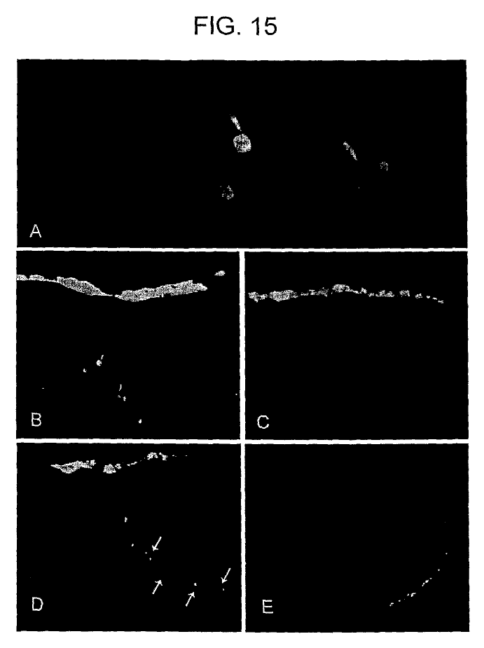

LV vectors pseudotyped with VSV glycoproteins were characterized in the context of the adult and postnatal developing mouse retina for comparison to RRV pseudotyped vectors. Following subretinal injection of VSV-CMV-GFP or VSV-CPA-GFP LV vectors in adult rats, a large surface area of the retina was observed to express GFP (FIG. 14). A developmental window was found to exist for the VSV-LV vector's ability to transduce photoreceptors when subretinally injected into C57BL/6 mice. This vector resulted in widespread expression in RPE cells in rodents of all ages, however transduction of photoreceptors occurred only in mice aged P7 and younger (FIG. 15). Importantly, at no time were GFP positive Müller cells observed with VSV-CMV-GFP LV vectors delivered to the retina. The temporal window for photoreceptor transduction coincides with a period of rod photoreceptor neurogenesis during retinal development (Carter-Dawson and M. M. LaVail, J Comp Neurol. 188(2), 263-272 1979). The onset of RPE specific transduction coincides with the completion of photoreceptor development and the beginning of normally occurring photoreceptor death (K. Mervin and J. Stone, Exp Eye Res. 75(6), 703-713 2002).

Accordingly, the results show that the vectors can specifically target Müller cells of the retina.

### Example 2

### CD44, GFAP, and Vim Promoters Drive GFP Expression in Müller Cells In Vivo

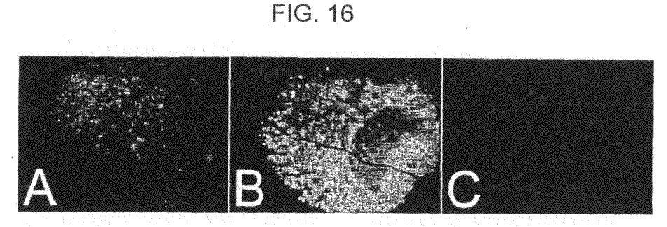

High titer vectors or PBS controls were injected subretinally or intravitreally into SD and S334Ter+/−rat eyes. GFP expression was evaluated by fluorescent fundus imaging 2-180 days following subretinal injection of 3 μl LV vector. GFP was observed over a 6 month period, showing persistent transgene expression and stable proviral integration. After subretinal injection of CD44, GFAP and Vim promoted vectors, high level GFP was consistently seen by fundus imaging (FIG. 16), and confocal microscopy revealed Müller cells were transduced with an efficiency approaching 95% in the subretinal bleb area (FIG. 17).

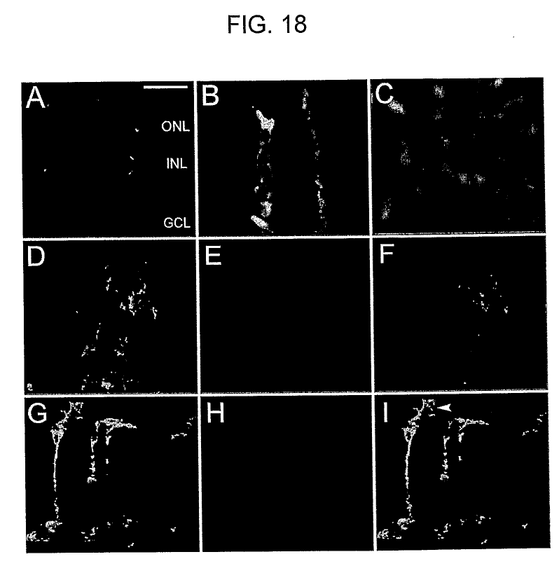

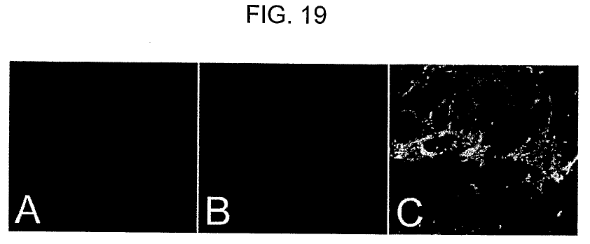

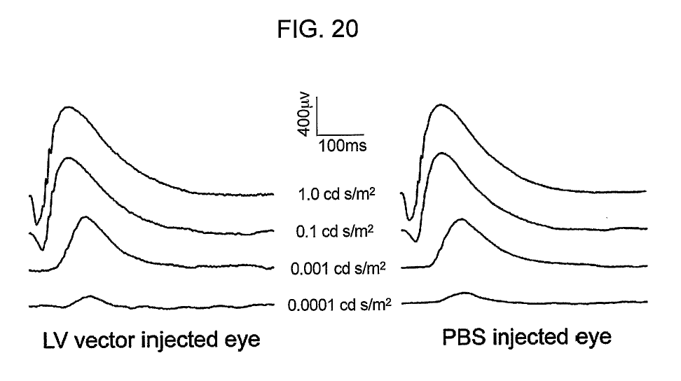

Overall fluorescence intensity viewed by fundus imaging consistently appeared highest with the CD44 promoter, followed by GFAP, and finally Vim promoted vectors. VSV.CD44.GFP vector injected retinas exhibited GFP expression in Müller cell processes spanning the entire retinal thickness (FIG. 18, panel A). Detailed Müller cell anatomy including processes enveloping photoreceptor cell bodies (FIG. 18, panel B) and the complex fiber basket matrix at the OLM are also observed en face (FIG. 18, panel C). GFP positive sections were immunostained with a Müller cell specific glutamine synthetase (GS) antibody, which exhibited co-localization with LV delivered GFP (FIG. 18, panels D-F). Following injection of VSV.GFAP.GFP vector in the S334Ter+/−degenerating retina, GFP positive Müller cells can be seen penetrating the OLM and invading the subretinal space of rhodopsin stained photoreceptor outer segments (FIG. 18, panels G-I). Obvious signs of reactive gliosis and glial scar formation can be observed in GFP positive Müller cells of degenerating S334Ter+/−retinas two months after injection (FIG. 19, panels A-C). Although predominant expression was seen in Müller cells in both SD and S334Ter retinas, some “leaky” GFP expression was observed in adjacent RPE cells. Vectors containing CMV, CAG, and ubiquitin-C promoters drove GFP expression solely in the RPE when injected subretinally in adult rats (FIG. 20). All vectors were also injected intravitreally (5-10 μl) in an attempt to transduce Müller cell endfeet at the ILM, however this delivery approach proved unsuccessful due to an unidentified barrier to LV vectors (FIG. 16, panel C).

### Example 3

### Photoreceptor Rescue by GDNF Secretion from LV Transduced Müller Cells

The present invention is used for treatment of multiple neurodegenerative diseases of the retina (i.e. RP, AMD, glaucoma). The neurotrophin GDNF has significant application in the treatment of RP and has been shown to delay photoreceptor degeneration when expressed in photoreceptors of the S334Ter-4 transgenic rat model for RP (Sanfter et al. Molec Ther, 4, 1-9, 2001).

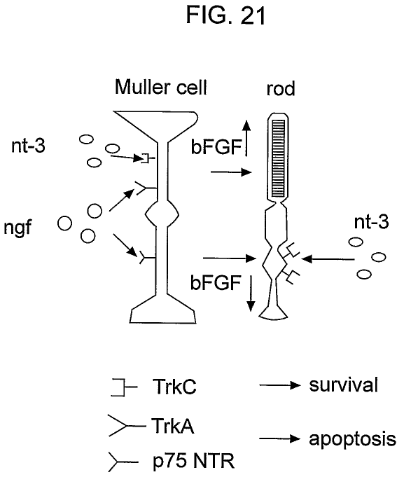

The secretion of neurotrophins from Müller cells directly to rescue degenerating photoreceptors has advantages over previous methods for neurotrophin rescue; Müller cells are not directly affected by known gene defects resulting in retinal degenerations and are therefore healthy reservoirs capable of secreting protective factors. Their unique retinal anatomy permits vector access from either intravitreal or subretinal injection. Furthermore, their close association with all other classes or retinal neurons insures the secreted factor will be delivered to the appropriate target cell. Müller cells are known to mediate photoreceptor survival in the light damage model for photoreceptor degeneration (Harada et al. Neuron. May; 26(2):533-41 2000). Müller cells have the innate ability to secrete endogenous growth factors that promote photoreceptor survival during times of insult or disease (FIG. 21).

### GFAP-GDNF Transfer Vector Design and LV Construction

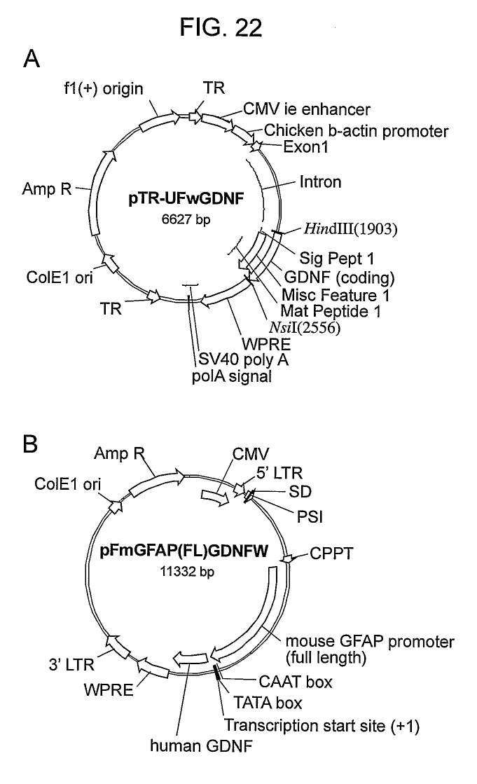

Vectors are based upon those described above in Example 1 demonstrating Müller cell specific expression. For construction of mGFAP(FL)-GDNF, the human GDNF (636 bp) cDNA is released from pTR-UFwGDNF by HindIII-NsiI restriction digest (FIG. 22, panel A). The GFP cDNA is excised from pFmGFAP(FL)GW by XbaI digest and the GDNF cDNA is blunt end ligated in place of GFP to create pFmGFAP(FL)GDNFW (FIG. 22, panel B). LV vectors containing the human GDNF cDNA expressed under control of a Müller specific, promoter (i.e. GFAP, Vimentin, CD44) are packaged as described above with envelope glycoproteins RRV or VSV. Packaged vector is delivered either subretinally or intravitreally to appropriate animal models for retina disease (i.e. S334Ter) and efficacy is determined by retinal thickness measures and ERG as described above.

### Example 4

### RRV Pseudotyped LV Vectors with Enhanced Heparin Binding Affinity

Müller cell transduction efficiency can be significantly increased when the vectors are delivered via the relatively non-invasive intravitreal injection approach. Certain viruses such as AAV2 are known to bind to heparan sulfate as its primary receptor and FGFR and an integrin as secondary receptors (Summerford & Samulski J Virol 72, 1438-45 1998, Qing et al. Nat Med 5, 71-7 1999, and Summerford et al. Nat Med 5, 78-82 1999). Heparan sulfate binding however, is not a recognized route for LV vector binding and cellular entry. The use of heparin sulfate as a moiety for LV vector attachment offers increased efficiency of Müller cell transduction as it is known that Müller cells express significant amounts of heparin sulfate on their endfeet at the ILM (inner limiting membrane) (Liang et al., Adv Exp Med Biol 533, 439-45 2003). The endogenous expression of heparin sulfate at the ILM is utilized for specific LV vector entry into Müller cells when vector is delivered via an intravitreal injection.

### Vector Design

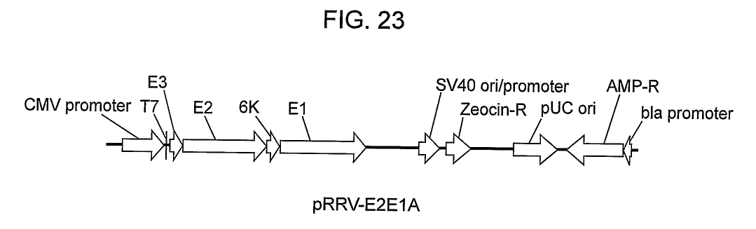

The RRV envelope glycoprotein is synthesized as a polyprotein that is processed into individual subunits. E2 and E1 form a heterodimer, both transmembrane proteins (Sharkey et al. JVI 75.6.2653-2659 2001). Eighty of these complexes (spikes) are found in the alphavirus envelope. The viral transmembrane glycoprotein complex is responsible for the binding of the alphavirus to the surface of a susceptible cell and for the fusion of the viral and cellular membranes that occurs during the process of viral entry. It consists of a trimer of heterodimers, with the heterodimer composed of two transmembrane proteins, E1 and E2 (FIG. 23).

Specific mutations introduced into the E2 region of the RRV envelope enable the RRV enveloped virus to use heparan sulfate as an attachment site for subsequent entry (Heil et al. JVI 75.14.6303-6309 2001). The results show that amino acid 218 of the E2 glycoprotein can be modified to create a heparan sulfate binding site and this modification expands the host range of Ross River virus in cultured cells to cells of avian origin. Of significant relevance to the present invention is the prevalence of heparan sulfate proteoglycans expressed on the cell surface of Müller cell endfeet at the ILM.

### Modified Envelope Glycoprotein Construction

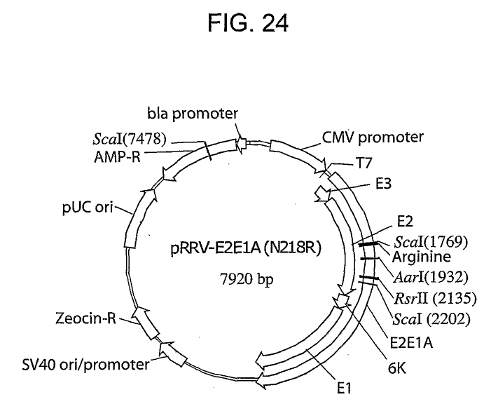

RRV envelope glycoprotein expression constructs harboring specific mutations at amino acid 218 were generated as follows. The RRV expression plasmid pRRV-E2E1A was digested with BsaBI-BlpI enzymes to remove the 1926 bp fragment of E2. Similarly, the RRV-N218R clone harboring the N218R mutation was digested with BsaBI-BlpI to release the mutated fragment. The fragment containing the N218R substitution was then ligated in place of the E2 region in pRRV-E2E1A to create pRRV-E2E1A(N218R) (FIG. 24). LV vectors containing Müller specific promoters and desired transgenes are packaged and injected intravitreally as described above.

The preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

That which is claimed is:

1. A method of treating or preventing diseases of the eye, comprising, administering to a subject a Müller cell specific retroviral gene delivery vector which directs the expression of a therapeutic polypeptide in the Müller cell, such that the disease of the eye is treated or prevented.

2. The method according to claim 1 wherein the disease of the eye is macular degeneration, diabetic retinopathy, retinitis pigmentosa, glaucoma, a surgery-induced retinopathy, retinal detachment, a photic retinopathy, a toxic retinopathy, or a trauma-induced retinopathy.

3. The method according to claim 1 wherein the therapeutic polypeptide is a neurotrophic factor.

4. The method according to claim 3, wherein the neurotrophic factor is FGF, NGF, BDNF, CNTF, NT-3, or, NT-4.

5. The method according to claim 1, wherein the therapeutic polypeptide is an anti-angiogenic factor.

6. The method according to claim 5, wherein the anti-angiogenic factor is soluble Flt-1, PEDF, soluble Tie-2 receptor, or, a single chain anti-VEGF antibody.

7. The method according to claim 1, wherein the therapeutic polypeptide is a neurotrophic factor.

8. The method according to claim 7, wherein the neurotrophic factor is GDNF, FGF, NGF, BDNF, CNTF, NT-3, or, NT-4.

9. The method of claim 1, wherein the vector is administered to the eye of the subject.

10. The method of claim 9, wherein the administering is by intraocular administration.

11. The method of claim 9, wherein the administering is by subretinal administration.

12. The method of claim 1, wherein the retroviral gene delivery vector is a lentiviral vector.

13. The method of claim 12, wherein the lentiviral vector is pseudotyped with a Ross River Virus glycoprotein.

14. A kit adapted for use in the method of claim 1, the kit comprising:
a sterile container containing a Müller cell specific retroviral gene delivery vector adapted for expression of a therapeutic polypeptide in an eye of the subject.

15. The kit of claim 14, wherein the kit comprises a sterile needle adapted for injection of the recombinant gene delivery vector into an eye of the subject.
